(19) United States
(12) Patent Application Publication
(10) Pub. No.: US 2008/0139497 A1
(43) Pub. Date: Jun. 12, 2008

(54) Use of ATP in controlled regional reperfusion as treatment during acute myocardial infarction

(76) Inventor: Jenchen Yang, Diamond Bar, CA (US)

Correspondence Address: ROGER A. GILCREST, 250 WEST STREET, COLUMBUS, OH, 43216-7513, US

(21) Appl. No.: 11/636,169

(22) Filed: Dec. 8, 2006

(51) Int. Cl. A61K 31/7076; A61P 9/10

(52) U.S. Cl. 514/47

(57) ABSTRACT

The invention is a method for controlled regional reperfusion using ATP-MgCl₂ after percutaneous coronary revascularization for acute myocardial infarction, the method comprising the step of administering an effective amount of ATP-MgCl₂ to the step of administering an effective amount of ATP-MgCl₂ to the infarct-related vessel(s), such as at a dosage level of at least 0.03 mg/kg/min. The method also includes a method for controlled regional reperfusion using ATP-MgCl₂ after percutaneous coronary revascularization for acute myocardial infarction, the method comprising the steps of (a) performing cardiac catheterization and coronary angiogram; (b) identifying the infarct-related vessel; (c) performing a left ventriculogram and calculating the left ventricular ejection fraction; and (d) performing a percutaneous coronary intervention; and after percutaneous revascularization of the infarct related vessel, infusing ATP-MgCl₂ intracoronary, preferably at a rate of at least 0.03 mg/kg/min through the balloon catheter.

### FIELD OF THE INVENTION

The invention relates generally to the area of treatment and/or reduction of infarct size attendant to myocardial infarction reperfusion. The beneficial effect of the invention is achieved through the use of pharmaceutical compositions that contain ATP-MgCl₂ to reduce infarct size at the site of reperfusion during myocardial infarction.

### BACKGROUND OF THE INVENTION

ST-elevation myocardial infarction (STEMI) continues to be a significant public health problem in industrialized countries and is becoming an increasingly significant problem in developing countries. It has been established that early coronary reperfusion during acute myocardial infarction salvages jeopardized myocardium and reduce infarct size.^{1,2,3} One method for establishing reperfusion, thrombolytic therapy, has been shown to improve survival in recent clinical trials.^{4,5,6} However, thrombolytic therapy is limited by its perceived or definite contraindications, intracranial bleeding, inability to establish Thrombosis In Myocardial Infarction (TIMI-3) flow in many patients, and high rates of recurrent ischemia and re-occlusion. Percutaneous coronary intervention (PCI) has been used in an effort to overcome the limitations of thrombolytic therapy. Certainly a preference for PCI has emerged in recent years.⁷ One of the articles by Keeley and Grines, for example, was a meta-analysis of 23 randomized trials suggesting superiority of catheter-based reperfusion over fibrinolytic therapy for the treatment of ST-elevation myocardial infarction (STEMI).⁸

It is clearly demonstrated that the etiology of acute myocardial infarction is caused by occlusive thrombus in the majority of cases. The myocardium supplied by the vessel distal to the occlusion becomes ischemic. In the ischemic state there is a gradation of cardiac muscle injury and a sequence of functional loss.⁹ In animal studies on coronary occlusion, an immediate cellular leak of K⁺ occurs and the rate of relaxation declines. Within one to two minutes, there is a complete loss of contraction followed by the onset of contracture in seven to ten minutes in isolated preparations. The major problem of this initial period, if the occlusion zone is not too great, is electrical dysfunction. The next 1 to 6 hours is the period of variable reversible injury. Depending on the degree of collateral circulation, this period can even be extended up to 24 hours; which may be the reason for survival benefit of thrombolytic therapy up to 24 hours after the onset of symptoms in the ISIS-II study.⁵

Myocardial ischemia results in rapid depletion of adenosine 5′-triphosphate (ATP), the universal high energy compound which is required for various metabolic processes. Although mechanical function ceases when ATP concentration remains high (˜50%), the initial decline in cardiac function and loss of ATP appears related.

Studies from a number of laboratories have shown that infusion of ATP-MgCl₂ proved beneficial for the survival of animals after hemorrhagic shock,^{10,11,12,13} severe burns,¹⁴ sepsis-peritonitis,¹⁵ post-ischemic hepatic failure,^{10,16} and endotoxin shock.^{17,18} Moreover, ATP-MgCl₂ has been shown to accelerate the recovery of renal function after acute renal failure in rats¹⁹ as well as mini-pigs.²⁰ In addition, it has been shown that kidneys that were subjected to episodes of warm ischemia could be salvaged by addition of ATP-MgCl₂ to the perfusate.²¹ ATP-MgCl₂ has also been effective in hastening renal recovery from a toxic injury.¹⁰ Kraven et al²² have shown that infused ATP-MgCl₂ decreased tissue lactate production, and they suggested that this was due to a direct intracellular effect of administered ATP. Moreover, these investigators²³ also showed that the treatment of animals in shock with ATP-MgCl₂ returned the altered member permeability toward normal. Machiedo et al²⁴ reported that exogenously administered ATP-MgCl₂ can reverse the inhibition of ornithine metabolism and the change in tissue lactate level during hemorrhagic shock. Since both of these are intracellular ATP-dependent reactions, this led them to conclude that ATP-MgCl₂ administration after hemorrhagic shock either replenishes intracellular ATP levels or returns the altered cell membrane permeability toward normal or both. In addition, ATP-MgCl₂ is being used in Japan for the treatment of acute renal failure.²⁵ ATP-MgCl₂ is also given to hepatectomy, sepsis-peritonitis, and acute hepatic failure patients.

Myocardial protection during surgically induced ischemia is provided by infusion of cardioplegic solutions which are designed to decrease energy requirements that will minimize cellular injury. Whether the degree of cellular injury is reversible or not is multifactorial, and recent evidence suggests that conditions of reperfusion are a major determinant of reversibility. A number of investigators have consequently tried to alter the reperfusion conditions so as to provide an environment which would allow the cellular reparative process to proceed as efficiently and rapidly as possible, while additional injury is avoided. Those reperfusion conditions involved providing an initial reperfusate administered under carefully controlled conditions which is low in calcium, high in osmolarity, and contains such additives as calcium channel blockers, oxygen free radicals scavengers and glucose. Under certain conditions of reperfusion, functional recovery has improved. Fedelesova et al²⁶ demonstrated that ATP injected into isolated nonperfused hypothermic dog hearts improved nucleotide and phosphocreatine levels. Furthermore, analysis of the intra-and extra-cellular distribution of nucleotides using ¹⁴C- and ³²P-labeled ATP demonstrated that a portion of the ATP entered the cell. Ziegelhoffer et al ²⁷ showed that a small amount of exogenously administered ATP but not ADP or AMP increased the ATP and total adenine nucleotide content of hypoxic myocardium. In a global ischemic model in the intact dog heart, McDonagh et al ²⁸ demonstrated improved myocardial recovery following normothermic ischemia with infusion of low dose ATP-MgCl₂. Kopf et al ^{29,30} also demonstrated that infusion of ATP- MgCl₂ can improve myocardial performance following prolonged ischemia.

Whether the ATP-MgCl₂ molecule can cross the plasma membrane and entered the cell remains controversial. It has been assumed that because of its highly polar nature with three negative charges, ATP cannot cross the plasma membrane. When ATP is complex with MgCl₂, it has one instead of three negative charges. In addition, the cell membrane is known to be permeable to macromolecules following ischemia.³¹ Buchthal et al³² demonstrated that externally added ATP induced contraction in isolated muscle fibers and suggested that ATP had permeated the cell membranes. In another study by Williams et al ³³ the addition of ATP to cultured myocardial cells caused an increase in ATP content and this effect was not due to breakdown products of ATP since neither adenosine nor AMP produced this effect.

### SUMMARY OF THE INVENTION

The purpose of the present invention is to provide controlled regional reperfusion using ATP-MgCl₂ after percutaneous coronary revascularization for acute myocardial infarction in order to reduce infarct size, improve left ventricular systolic function, and improve survival.

The present invention includes a method of treating myocardial infarct through controlled regional reperfusion. This regional reperfusion is preferably preformed at or near the site of any percutaneous intervention procedure related to treatment, such as the site of balloon angioplasty and the insertion of a stent. Typically, the regional reperfusion is carried out by direct arterial infusion to an open artery, and preferably within a short time (typically right after the percutaneous revascularization procedure related to treatment).

In general terms, the present invention includes a method for controlled regional reperfusion using ATP-MgCl₂ after percutaneous coronary revascularization for acute myocardial infarction, the method comprising the step of administering an effective amount of ATP-MgCl₂ to the infarct-related vessel. The method of the present invention involves administering an effective amount of ATP-MgCl₂ to an artery of said body of at a dosage sufficient to reduce ischemia. It is preferred that the dosage of the ATP-MgCl₂ is at least 0.03 mg/kg/min.

The method for controlled regional reperfusion using ATP-MgCl₂ after percutaneous coronary revascularization for acute myocardial infarction, also may include steps of: (a) performing cardiac catheterization and coronary angiogram; (b) identifying the infarct-related vessel; (c) performing a left ventriculogram and calculating the left ventricular ejection fraction; and (d) performing a percutaneous coronary intervention; and after percutaneous revascularization of the infarct related vessel, infusing ATP-MgCl₂ intracoronary via balloon catheter at a rate of at least 0.03 mg/kg/min.

The method of the present invention may be used to reduce the effects of ischemia to other organs, such as the brain, liver or kidneys.

### DETAILED DESCRIPTION OF THE PREFERRED EMBODIMENT

In accordance with the foregoing summary, the following presents a preferred embodiment of the present invention which is presently considered to be the best mode thereof.

### EXAMPLE PROTOCOL

Patients presented with acute myocardial infarction clinically may be taken to the catheterization laboratory. Cardiac catheterization and coronary angiogram then may be performed in the standard fashion. The infarct related vessel may thereby be identified clinically and angiographically. A left ventriculogram is then to be performed and the left ventricular ejection fraction will be calculated by the area-length method from the right anterior oblique projection of the left ventriculogram before percutaneous coronary intervention. After successful percutaneous revascularization of the infarct related vessel, the guide wire will then be withdrawn from the balloon catheter and infusion of ATP-MgCl₂ at a rate of 0.03 mg/kg/min is then performed for about 30 minutes via the central lumen of the balloon catheter. Heart rate, blood pressure, pulmonary capillary wedge pressure, and cardiac output then may be monitored pre- and post- infusion of ATP- MgCl₂. It is preferred that the ATP- MgCl₂ is 99% pure.

Follow up left ventricular function study by echocardiogram may then be performed within one week or prior to hospital discharge and at six months. Long-term follow up for MACE (recurrent angina, MI, and death) may be carried out thereafter at an appropriate cardiology clinic.

The administration of ATP- MgCl₂ in accordance with the present invention typically and preferably will be done in accordance with percutaneous intervention at the affected site, such as the placement of a stent or application of balloon angioplasty to the affected area. The ATP- MgCl₂ is directly infused into an artery, such as the infarct related coronary artery, which best prevents its breakdown and allows it to be effective at the affected site. It may be directly infused in solution preferably for approximately 10 to 30 minutes after percutaneous intervention, and may be done using the same balloon catheter.

Preferably, the ATP- MgCl₂ will be in the form of a buffered solution at physiologic pH, such through use of a phosphate buffer in saline.

### Safety of Administering ATP-MgCl₂

ATP-MgCl₂ has been used for intravenous infusion into human subjects under various conditions in Japan and Europe. ATP has also been used as an intravenous bolus up to a maximum of 60 mg for treatment of supraventricular tachycardia.³⁴ In the United States, the safety and hemodynamic response of ATP-MgCl₂ in man has been demonstrated by Chaudry et al.³⁵ Also, ATP-MgCl₂ has been infused into the left coronary artery in patients with coronary artery disease with reduction of myocardial oxygen consumption in the absence of changes in the measured determinants of myocardial oxygen demand. This finding suggests a possible oxygen sparing effect of ATP.^{36,37}

Accordingly, direct regional reperfusion using ATP-MgCl₂ is likewise a safe method of treatment for acute myocardial infarction, as well as for treating other conditions where ischemia may occur.

### Discussion

The state of the art paper by Kloner and Rezkalla³⁸ summarized elegantly the past and current approach of cardiac protection during acute intervention or surgery. The concept of glucose-insulin-potassium infusion provides substrates to increase glycolytic ATP (adenosine triphosphate) synthesis during reperfusion is a reasonable idea³⁹, but more direct approach is to provide the universal energy source directly by infusion of ATP-MgCl₂ during acute intervention, as is done in accordance with the present invention. ATP-MgCl₂ treatment after experimental acute myocardial ischemia protects the heart from the adverse effects of ischemia.⁴⁰ ATP-loaded liposomes effectively protected the ischemic heart muscle in rabbits with an experimental myocardial infarction as evidenced by a significantly decreased fraction of the irreversibly damaged heart within the total area at risk.⁴¹

### REFERENCES

The following references are hereby incorporated herein by reference:
1. Reimer K A, Lowe J E, Rasmussen M M, Jennings R B. The wave front phenomenon of ischemic cell death. 1. Myocardial infarct size versus duration of coronary occlusion in dogs. Circulation 1977;56:786-94.
2. Theroux P, Ross J, Franklin D, Kemper W S Sasayama S. Coronary artery reperfusion. III Early and late effects on regional myocardial function and dimensions in conscious dogs. Am J Cardiol 1976;38:599-606.
3. Stack R S, Phillips H R, Grierson D S et al. Functional improvement of jeopardized myocardium following intracoronary streptokinase infusion in acute myocardial infarction. J Clin Invest 1983;72:84-95.
4. Gruppo Italiano per lo Studio della Streptochinasi nell'Infarto Miocardio (GIS SI): Long-term effects of intravenous thrombolysis in acute myocardial infarction: Final report of the GIS SI study. Lancet 1987;2:871-4.
5. ISIS-2 (Second International Study of Infarct Survival) Collaborative Group: Randomized trial of intravenous streptokinase, oral aspirin, both or neither among 17,187 cases of suspected acute myocardial infarction: ISIS-2. Lancet 1988;1:349-60.
6. Fibrinolytic Therapy Trials' (FTT) Collaborative Group. Indications for fibrinolytic therapy in suspected acute myocardial infarction: collaborative overview of early mortality and major morbidity results from all randomized trials of more than 1000 patients. Lancet. 1994;343:311-322.
7. Grines C L, Browne K F, Marco J, et al. A comparison of immediate angioplasty with thrombolytic therapy for acute myocardial infarction. N Engi J Med. 1993;32:673-679.
8. Keeley E C, Boura J A, Grines C L. Comparison of primary angioplasty and intravenous thrombolytic therapy for acute myocardial infarction: a quantitative review of 23 randomized trials. Lancet. 2003;361:13-20.
9. Langer G A, Weiss J N, Schelbert H R. Cardiac ischemia. Part I—Metabolic and physiologic response. West J Med 1987 June;146:713-723.
10.Chaudry I H, Clemens M G, Baue A E. Alterations in cell function with ischemia and shock and their correction. Arch Surg 1981;116:1309-1317.
11. DiStazio J, Maley W B, Thompson H, Sembrat S, Stremple J. Effect of ATP-MgCl₂-glucose administration during hemorrhagic shock on cardiac metabolism function and survival. Adv Shock Res 1980;3:153-166.
12. Koc E A, Diakoumis K N, Karl R C. Effect of ATP-MgCl₂ on plasma glucose, insulin and glucagon levels in rats subjected to hemorrhagic shock. Surg. Forum 1980;31:8-10.
13. Kraven T, Rush B F, Ghosh A, Adams-Griffin M. Improved survival and metabolic changes in a rat shock model produced by ATP-MgCl₂. Curr. Surg. 1979;36:435-437.
14. Zaki M S, Burke J L, Trelstad R L. Protective effect of adenosine-triphosphate administration in burns. Arch. Surg. 1978;113:605-610.
15. Chaudry I H, Clemens M G, Ohkawa M, Schleck S, Baue A E. Restoration of hepatocellular function and blood flow following ischemia with ATP-MgCl₂. Adv. Shock Res. 1982;8:177-186.
16. Hirasawa H, Ohkawa M, Odaka M, Sato H. Improved survival, RES function and ICG clearance tests with ATP-MgCl₂ following hepatic ischemia. Surg. Forum 1979;30:158-160.
17. Filkins J P, Buchanan B J. Protection against endotoxin shock and impaired glucose homeostasis with ATP. Circ. Shock 1977;4:253-258.
18. Fulton R L. Prevention of endotoxin death with nicotinamide and adenosine triphosphate. Surg. Forum 1974;25:17-19.
19. Siegel N J, Glazier W B, Chaudry I H, Gaudio K M, Lytton B, Baue A E, Kashgarian M. Enhanced recovery from acute renal failure by post-ischemic infusion of adenine nucleotides and magnesium chloride in rats. Kidney Int. 1980;17:338-349.
20. Lytton B, Glazier W B, Chaudry I H, Baue A E. The use of adenosine triphosphate with magnesium chloride in the treatment of post-ischemic renal injury. Trans Am Assoc Genitourin Surg. 1978;70:145-8.
21. Lytton B, Vaisbort V R, Glazier W B, Chaudry I H, Baue A E. Improved renal function using ATP-MgCl₂ in preservation of canine kidneys subjected to warm ischemia. Transplantation 1981;31:187-189.
22. Kraven T, Rush B, Slotman G J, Adams-Griffin M. Permeability of the shock cell to ATP-MgCl₂. Surg. Forum 1979;30:7-9.
23. Kraven T, Rush B, Ghuman S S, Dikdan G S. Reversal of altered permeability of the shock cell to ATP-MgCl₂. Surg. Forum 1980;31:3-5.
24. Machiedo G W, Ghuman S, Rush B F, Kraven T, Dikdan G S. The effect of ATP-MgCl₂ infusion on hepatic cell permeability and metabolism following hemorrhagic shock. Surgery 1981;90:328-335.
25. Odaka M, Hirasawa H, Tabata Y, Kobayashi H, Sato H. A new treatment of acute renal failure with direct hemo-perfusion enhancement of reticuloendothelial system and ATP-MgCl₂. Satellite Symp. Acute Renal Failure, Tel Aviv, Israel, June 1981, p. 125.
26. Fedelesova M, Ziegelhoffer A, Krause E, Wollenberger A. Effect of exogenous adenosine triphosphate on the metabolic state of the excised hypothermic dog heart. Circ. Res. 1969;24:617-27.
27.Ziegelhoffer M, Fedelesova A, Kostolansky S. Specific ATP action on the metabolism of isolated heart: Influence of pH, divalent cation concentration and stability of complexes. Acta Biol. Med. Ger. 1972;28:893.
28. McDonagh P F, Laks H, Chaudry I H, Baue A E. Improved myocardial recovery from ischemia. Arch. Surg. 1984;119:1379-84.
29. Kropf G S, Chaudry I H, Condos S G, Baue A E. Improved myocardial performance after prolonged ischemia with ATP-MgCl₂ cardioplegia. Surg. Forum 1986;37:234.
30. Kropf G S, Chaudry I H, Condos SG, Baue A E. Reperfusion with ATP-MgCl₂ following prolonged ischemia improve myocardial performance. J Surg Res 1987;43:114-117.
31. Higgins T J, Bailey P J. The effect of cyanide and iodoacetate intoxication and ischemia on enzyme release from the perfused rat heart. Biochim Biophys Acta. 1983;762:67-75.
32. Buchthal F, Deutsch A, Knappic G. Further investigation on the effect of adenosine triphosphate and related phosphorous compounds on isolated striated muscle fibers. Acta Physiol. Scand. 1946;11:325.
33. Williams D, Ribell D, Rovetto M J. ATP induced increase in ATP content of cultured myocardial cells. Fed. Proc. 1979;38:1389.
34. Viskin S, Fish R, Glick A, Glikson M, Eldar M, Belhassen B. The adenosine triphosphate test: A bedside diagnostic tool for identifying the mechanism of supraventricular tachycardia in patients with palpitation. J Am Coll Cardiol 2001;38:173-7.
35.Chaudry I H, Keefer J R, Barash P, Clemens M G, Kropf G S, Baue A E. ATP-MgCl₂ infusion in man: increased cardiac output without adverse systemic hemodynamic effects. Surg Forum 1984;35:14-16.
36. Wohigelernter D, Jaffe C, Cleman M, Young L, Clemens M, Chaudry I H. Effects of ATP-MgCl₂ on coronary blood flow and myocardial oxygen consumption. Circulation 1985;72:111-315.
37. Nanto S, Kitakaze M, Takano Y, Hori M, Nagata S. Intracoronary administration of adenosine triphosphate increases myocardial adenosine levels and coronary blood flow in man. Jpn Circ J. 1997 Oct;61(10):836-42.
38. Kloner R A, Rezkalla S H. Cardiac Protection During Acute Myocardial Infarction: Where Do We Stand in 2004? J Am Coll Cardiol 2004;44:276-86.
39. Opie L H, Sack M N. Metabolic plasticity and the promotion of cardiac protection in ischemia and ischemic preconditioning. J Mol Cell Cardiol 2002;34:1077-1089.
40. Katircioglu S F, Ulus A T, Saritas Z. ATP-MgCl₂ treatment after experimental acute myocardial ischemia and reperfusion. Panminerva Med. 2000 Mar;42(1):11-5.
41. Verma D D, Hartner W C, Levchenko T S, Bernstein E A, Torchilin V P. ATP-loaded liposomes effectively protect the myocardium in rabbits with an acute experimental myocardial infarction. Pharmaceutical Research 2005;22:2115-2120.

Other embodiments and configurations may be devised without departing from the spirit of the inventions and the scope of the appended claims.

What is claimed is:

1. A method for controlled regional reperfusion of a body using ATP-MgCl₂ after percutaneous coronary revascularization for acute myocardial infarction, said method comprising the step of administering an effective amount of ATP-MgCl₂ to an artery of said body of at a dosage sufficient to reduce infarct size.

2. A method according to claim 1, wherein the dosage of said ATP-MgCl₂ is at least 0.03 mg/kg/min.

3. A method for controlled regional reperfusion of a body using ATP-MgCl₂ after percutaneous coronary revascularization for acute myocardial infarction, said method comprising the step of providing balloon angioplasty or a stent to the site of said myocardial infarction prior to administering an effective amount of ATP-MgCl₂ to an artery of said body of at a dosage sufficient to reduce ischemia.

4. A method for controlled regional reperfusion using ATP-MgCl₂ after percutaneous coronary revascularization for acute myocardial infarction, said method comprising the steps of (a) performing cardiac catheterization and coronary angiogram; (b) identifying the infarct-related vessel; (c) performing a left ventriculogram and calculating the left ventricular ejection fraction; and (d) performing a percutaneous coronary intervention; and after percutaneous revascularization of the infarct related vessel, infusing ATP-MgCl₂ at a rate of at least 0.03 mg/kg/min through the balloon catheter.

5. A method for treating or preventing ischemia attendant to myocardial infarction in a patient, comprising administering to said patient an effective amount of ATP-MgCl₂ to reduce infarct size in said patient brought about by myocardial infarction.
